(19) United States
(12) Patent Application Publication
(10) Pub. No.: US 2010/0004566 A1
(43) Pub. Date: Jan. 7, 2010

(54) INTELLIGENT ORTHOTIC INSOLES

(75) Inventors: Jae Son, Rancho Palos Verdes, CA (US); Glen D. Hinshaw, Scottsdale, AZ (US); David Ables, Los Angeles, CA (US)

Correspondence Address: GEORGE A. HERBSTER, 100 CUMMINGS CENTER, SUITE 213-C, BEVERLY, MA, 01915, US

(73) Assignee: ESOLES, L,L.C., Scottsdale, AZ (US)

(21) Appl. No.: 12/350,605

(22) Filed: Jan. 8, 2009

Related U.S. Application Data
(60) Provisional application No. 61/020,598, filed on Jan. 11, 2008.

(51) Int. Cl. A61B 5/103; A43B 13/38; G08B 1/08

(52) U.S. Cl. 600/592; 36/43; 340/539.11

(57) ABSTRACT

An intelligent insole for generating time sensitive information about the pressure on the foot. The insole includes a custom-made, semi-custom or generically sized orthotic component. The orthotic is laminated with a top cover and an intermediate pressure sensor having an array of capacitive pressure sensors. Signal processing equipment may be embedded in the insole or placed locally with the insole as on the side of a shoe. The processor also can connect to a wireless transmitter for relaying the information to a remote site.

### CROSS REFERENCE TO RELATED APPLICATION

This application claims benefit from U.S. Provisional Patent Application Ser. No. 61/020,598 filed Jan. 11, 2008 for an Intelligent Orthotic Insole.

This application cross references U.S. patent application Ser. No. 11/116,738 filed Apr. 28, 2005 for a Method and Apparatus for Manufacturing Custom Orthotic Footbeds and assigned to the same assignee as this invention, which application was granted on Jul. 1, 2008 as U.S. Pat. No. 7,392,559.

### BACKGROUND OF THE INVENTION

1. Field of the Invention

This invention generally relates to orthotic insoles and more particularly to an insole that dynamically produces pressure maps of an individual's foot.

2. Description of Related Art

Over the years greater emphasis has been placed on the need for athletic performance and individual exercise. Athletes need to understand the ramifications of change in their routines. For example, information about the pressure on the bottom of an athlete's foot is important to cyclists indicating cycle cadence, power and/or force and distance traveled. For runners knowledge of the power/force being exerted, balance and alignment, insole wear due to repetitive compression of running, cadence and distance traveled are important parameters. Golfers want to know that they remain balanced during a swing.

Other individuals who exercise want immediate and cumulative feedback as to the success of their efforts. Particularly, they may wish to have information about distance traveled, energy exerted, weight reduction and other matters.

At risk individuals suffering from diabetes or other nerve degenerative diseases may utilize information about continuous pressure “hot spots” in order to reduce the risk of pressure ulcers or sores. Other individuals suffering from balance disorders may also use balance information to assist their mobility.

In still another set of applications, the same set of performance information (force, balance, cadence, etc.) can control a virtual avatar in an entertainment system such as a video games console allowing the translation of physical movement to virtual movement.

There have been a number of proposals for measuring the pressure exerted by a foot. Some such suggestions are contained in the following patents:

| | | |
|---|---|---|
| 5,033,291 | (1991) | Podollof et al. |
| 5,449,002 | (1995) | Goldman |
| 5,678,448 | (1997) | Fullen et al. |
| 5,875,571 | (1999) | Huang |
| 6,505,522 | (2003) | Wilssens |
| 6,807,869 | (2004) | Farringdon et al. |
| 7,191,644 | (2007) | Haselhurst et al. |

The Podollof et al. patent discloses a flexible tactile sensor for measuring foot pressure distributions by means of pressure sensitive resistive sensors.

The Goldman patent discloses a capacitive biofeedback sensor that is integral with a shoe and includes a resilient polyurethane dielectric. The sensor can be placed in the patient's shoe, boot, ankle brace, crutch handgrip, wheel chair, etc. to provide biofeedback.

The Fullen patent discloses a system for continually measuring the forces applied to the foot. This is a self-contained system that positions a force sensor array within the individual's shoe between the foot and the inner sole of the shoe. The system also discloses a rigid substrate that requires portions to be removed so the structure conforms to the individual's foot.

In the Huang patent an insole pad has a step counting device in the form of a fluid bag and fluid pressure monitor.

The Wilssens patent discloses an apparatus and method for measuring the pressure distribution generated by a three-dimensional object for obtaining a total force applied.

In the Farringdon et al. patent a shoe-based force sensor is interposed between the foot and the ground in the region of the shoe's sole. The sensor is resistive and has a middle layer of a resilient compressible elastomeric middle layer of insulating material which includes embedded electrically conductive metal filaments.

The Haselhurst patent discloses an insole pressure sensor and personal annunciator used to assist in the therapeutic treatment of subjects who have difficulty walking. A removable insole is placed inside the shoe which proportionally senses the touch down of the limb, with the measurement being transmitted to a receiver for analysis.

The foregoing patents are representative of a number attempts to use pressure or force sensing on the bottom of the foot. However, each seems to require a separate element to provide the pressure measurements typically without an orthotic device. The body incorporating the sensor does not provide any other function. In this prior art, some sensors are built into the shoe itself and therefore are not transferable among shoes.

What is needed is an intelligent orthotic insole that dynamically maps the pressure of the foot and is adapted to process these maps for wireless transmission to a receiver and other analysis equipment.

### SUMMARY

Therefore it is an object of this invention to provide an integral orthotic insole that maps the pressure of the foot.

Another object of this invention is to provide an orthotic insole that maps the pressure of the foot and is adapted to process the pressure map to a final format for use.

In accordance with one aspect of this invention an intelligent orthotic insole comprises a lower portion for engaging the shoe surface and defining an orthotic surface for the individual's foot. A cover is substantially coextensive with the lower portion and overlies an intermediate force sensing matrix. The lower portion, cover and force sensing matrix are formed as an integral, removable inner sole. A processor receives the signals from the source sensing matrix to generate output signals corresponding to the location and magnitude of the pressure exerted by the foot. These signals can be transmitted to a remote location.

### BRIEF DESCRIPTION OF THE DRAWINGS

The appended claims particularly point out and distinctly claim the subject matter of this invention. The various objects, advantages and novel features of this invention will be more fully apparent from a reading of the following detailed description in conjunction with the accompanying drawings in which like reference numerals refer to like parts, and in which:

FIG. 1 is a bottom exploded view in perspective of an orthotic constructed in accordance with this invention;

FIG. 2 is a top exploded view in perspective of the orthotic of FIG. 1 showing a lower portion, a cover and an intermediate force sensing matrix;

FIG. 3 is a schematic view of a system incorporating this invention; and

FIGS. 4 through 6 are images of pressure maps generated by the feet of three individuals.

### DESCRIPTION OF ILLUSTRATIVE EMBODIMENTS

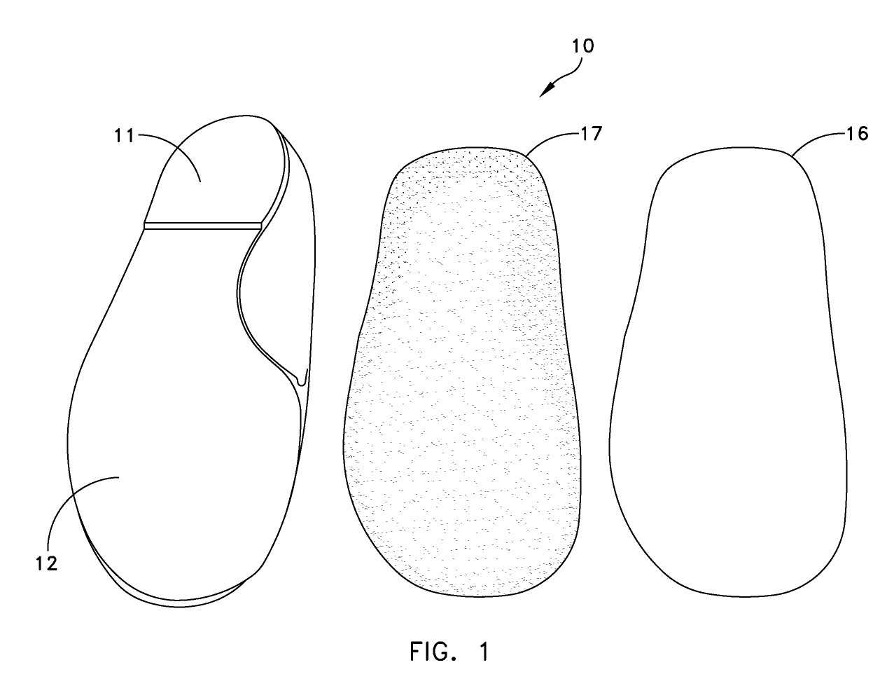

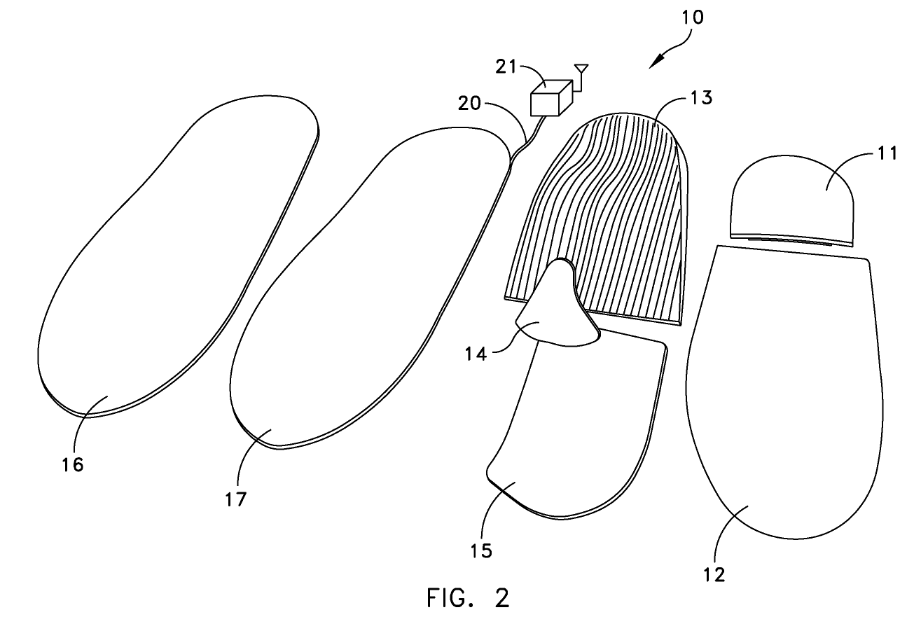

The above-identified U.S. Pat. No. 7,392,559 describes the construction of an orthotic insert without this invention. FIGS. 1 and 2 depict an intelligent orthotic insole 10 constructed in accordance with this invention with a heel post stabilizer 11 and a midfoot stabilizer 12 at a bottom layer. An orthotic 13 is constructed in accordance with U.S. Pat. No. 7,392,559 and lies on top of the stabilizers 11 and 12. The insole 10 may also include a metatarsal support 14 and a forefoot post 15. A top cover 16 overlies the entire orthotic insole 10. As known, the orthotic insert itself has some thickness in order to allow the machining to occur.

FIGS. 1 and 2 also depict an orthotic insole 10 with an integral sensor pad 17 that is a capacitive force sensing matrix that provides information about the magnitude and position of the pressure to produce a three-dimensional pressure map. For integration into an insole 10, the sensor 17 in FIGS. 1 and 2 preferably forms a flexible tactile sensor with an embedded array of capacitive sensors. There are several alternative embodiments. In some, first and second stand-alone thin layers contain conductor traces typically orthogonal to one another and lie on opposite sides of a dielectric material. The thin layers can comprise conductive ink traces on polyurethane or on an ethylene vinyl acetate (EVA) material, or on silicone molded on a polyurethane, EVA or trifluoropropolcarbonate (FPCB) substrate.

Other configurations use discrete sensor elements that are strategically located within the insole for monitoring force and balance. A first layer of conductive cloth, mesh or painted surface can be inserted or applied to a layer of thin compressible dielectric material such as EVA, silicone or polyurethane. A second layer on the opposite side of the dielectric material contains a circuit with electrodes where pressure or force is measured. The second layer of electrodes can be constructed using flexible circuit materials such as copper clad Kapton where the extraneous copper is etched away or using a conductive ink printing method on a Mylar film.

One advantage of using the same material that is used for the insole, such as EVA, is that the baseline value of the sensor itself can be a good indication of the insole wear as foam materials such as EVA compression set over time and use. It maybe necessary to design in geometric structures or patterns in the dielectric layer of the sensor to provide room for the material to compress thus amplifying the amount of deformation between the electrodes.

For a modular insole design where components of the insoles are selected based on the measured individual's anatomy, sensor elements can be placed inside the modular components and a provision to electrically connect the sensor element to the processor 21, can be achieved using a snap button or electrically conductive tape.

The use of a sensor array permits the construction of a sensor pad with a range of spatial resolutions. While any range up to 200 or more sensors is possible, it appears that a resolution from 3 to 20 sensors embedded in the sensor pad 17 will be sufficient.

Conductors 20 connect the sensing matrix in the sensor pad 17 to a processor 21 for monitoring the sensor outputs and converting them into time-stamped data packets on which a dynamic pressure map is based. In accordance with this invention the processor 20 may be embedded in the orthotic 13. The conductors 20 may also extend the processor 21 externally to be clipped to the individual's shoe such as the outer edge opening or some part of the individual's body, such as around the ankle.

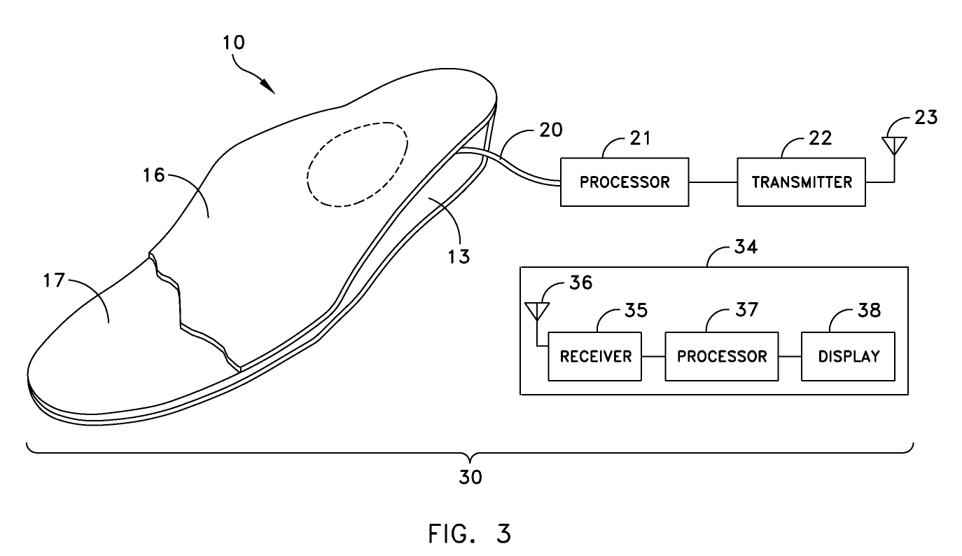

In a preferred form of this invention and as shown in FIG. 3, an insole 10 with a sensor pad 17 and processor 20 can be included in a system 30 with a transmitter 32 and antenna 33 that wirelessly transmits the data for reception in a remote receiver system 34 that includes a receiver 35 and antenna 36. A processor 37 produces an output for a display 38 in response to the received signals. This system 30 has the advantage of allowing data to be gathered dynamically at a remote location even as the individual is exercising. Alternatively, the processor may have sufficient storage to gather the data for subsequent download on demand.

Either a battery or motion-induced generator provides power for the processor 21 and transmitter 32. For example, the power supply can comprise a vibrating magnet generator that is integral with the processor 20.

The structure of the system of FIG. 3 provides flexibility in configuring the system and in determining the information that is transmitted. For example, if the transmitter 32 is Bluetooth capable, a modern cell phone can constitute the remote receiver system 34 in FIG. 3. The remote receiving system could also be capable of sending emails or providing auditory and/or tactile cues.

Different messages could be sent. A simplified text message or email might include a date, an activity level and a weight trend. A more technical message or email might include an activity level with a comparison to prior day's events and a quantification of the weight trend over the past several days or weeks.

Different auditory or tactile cues could provide real time feedback to the user depending on the application. For the golfer could use such feedback to indicate that his or her balance was too far forward. Feedback to a diabetic can indicate that too much pressure has been on the heel of one foot for too long. Balance-impaired individuals may benefit from an indication that one foot has made a solid contact when walking down a flight of stairs.

Because a person generally always wears shoes and performs majority of activities during this period, and since the intelligent insole can be placed into different shoes, continuous use of the intelligent insole is much easier than other activity monitoring devices such as pulse monitor with a chest trap or a shoe specific monitoring devices as described in the prior art patents.

An intelligent insole that wirelessly transmits performance data to a remote host can also be used as a controller for entertainment systems including video game consoles, mobile gaming devices, or mobile phones. The same information previously listed, including for example force cadence or balance can be used to translate user motion to virtual motion of an avatar.

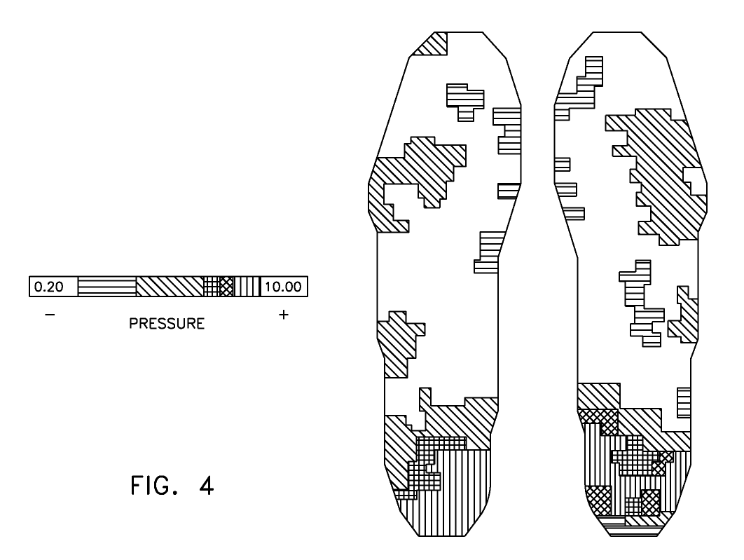

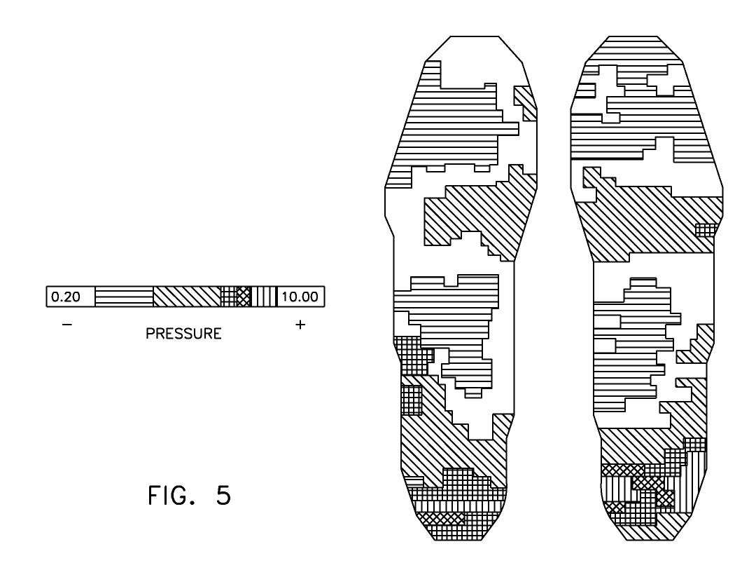

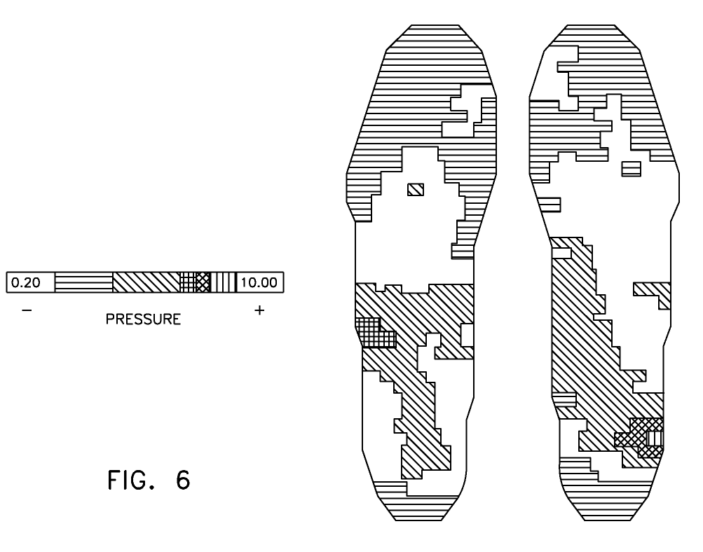

As will now be apparent, an intelligent insole, such as the insole 10 shown in FIGS. 1 and 2, used in a system such as shown in FIG. 3, can produce images such as those in FIGS. 4 through 6. These are screen shots taken during dynamic activities. It is clear that the sensor is identifying the pressure magnitude and location for the foot.

Thus a system constructed in accordance with this invention provides an orthotic insole that maps the pressure of a foot. It is adapted to process these pressure maps into a format for transmission over a wireless network for communication with a variety of devices including cell phones and for generating email messages.

This invention has been disclosed in terms of certain embodiments. It will be apparent that many modifications can be made to the disclosed apparatus without departing from the invention. For example, a sensor pad 17 integrated with the insole could be one of several versions. Sensors other than capacitive sensors can be substituted in an insole, such as resistive sensors, provided known problems with such sensors can be tolerated. Therefore, it is the intent of the appended claims to cover all such variations and modifications as come within the true spirit and scope of this invention.

What is claimed as new and desired to be secured by Letters Patent of the United States is:

1. An intelligent orthotic insole for insertion into an individual's shoe for broadcasting output signals that represent the pressure exerted by the individual's foot on said insole, said insole comprising:
A) an integral structure including:
i) a lower portion for engaging the shoe surface and defining a orthotic for the individual, and
ii) sensor pad means affixed to said lower portion for generating an array of signals containing information about the magnitude and position of pressure thereon,
B) a processor responsive to signals from said sensor pad means for generating corresponding output signals, and
C) means attached to said processor for transmitting the output signals to a remote location.

2. The intelligent orthotic insole as recited in claim 1 wherein said sensor pad means comprises a capacitive force sensing matrix that produces a three-dimensional pressure map of the forces acting on the insole.

3. The intelligent orthotic insole as recited in claim 2 wherein said capacitive force sensing matrix includes a compressible dielectric material.

4. The intelligent orthotic insole as recited in claim 2 wherein said sensor pad means is formed as a flexible tactile sensor.

5. The intelligent orthotic insole as recited in claim 2 wherein said sensor pad means comprises an embedded capacitive pressure sensor array.

6. The intelligent orthotic insole as recited in claim 2 wherein the sensor pad means comprises embedded discrete capacitive pressure sensors.

7. The intelligent orthotic insole as recited in claim 2 wherein said force sensing matrix comprises up to 200 pressure sensing locations.

8. The intelligent orthotic insole as recited in claim 2 wherein said force sensing matrix comprises between 3 and 20 pressure sensing locations.

9. The intelligent orthotic insole as recited in claim 2 wherein said processor converts the signals from the sensor pad means into additional time-stamped data packets.

10. The intelligent orthotic insole as recited in claim 2 wherein said processor is embedded in said integral structure.

11. The intelligent orthotic insole as recited in claim 10 wherein said transmitting means is embedded in said integral structure.

12. The intelligent orthotic insole as recited n claim 2 additionally comprising an integral cover overlying was lower portion and said sensor pad means.

13. An intelligent orthotic insole for providing time sensitive information about pressure on the foot comprising:
A) an integral insole structure including:
i) a lower portion for engaging the shoe surface and defining a orthotic for the individual,
ii) a cover substantially coextensive with the lower portion,
iii) sensor pad means attached to said lower portion and integral therewith for generating an array of signals containing information about the magnitude and position of pressure thereon,
B) a processor responsive to signals from said sensor pad means for generating corresponding output signals,
C) means attached to said processor for transmitting the wireless output signals, and
D) receiving means at a remote location for receiving the wireless output signals and displaying the results thereof.

14. The intelligent orthotic insole as recited in claim 13 wherein said receiving means comprises:
i) an antenna,
ii) a receiver for the wireless output signals,
iii) a display, and
iv) a processor for converting the received wireless output signals from the receiver into a display of the pressure exerted on the orthotic insole.

15. The intelligent orthotic insole as recited in claim 14 wherein said sensor pad means comprises a capacitive force sensing matrix that produces a three-dimensional pressure map of the forces acting on the insole.

16. The intelligent orthotic insole as recited in claim 14 wherein said capacitive force sensing matrix includes a compressible dielectric material.

17. The intelligent orthotic insole as recited in claim 14 wherein said sensor pad means is formed as a flexible tactile sensor.

18. The intelligent orthotic insole as recited in claim 14 wherein said sensor pad means comprises an embedded capacitive pressure sensor array.

19. The intelligent orthotic insole as recited in claim 14 wherein the sensor pad means comprises embedded discrete capacitive pressure sensors.

20. The intelligent orthotic insole as recited in claim 14 wherein said processor converts the signals from the sensor pad means into additional time-stamped data packets.
